(19) United States
(12) Patent Application Publication
(10) Pub. No.: US 2025/0001145 A1
(43) Pub. Date: Jan. 2, 2025

(54) DILATOR SHAFT DESIGN ENABLING TIP SHAPABILITY AND VARIABLE SHAFT FLEXIBILITY

(71) Applicant: BIOTRONIK AG, Buelach (CH)

(72) Inventors: Marc Gianotti, Wiesendangen (CH); Michael Jetter, Thayngen (CH)

(21) Appl. No.: 18/709,639

(22) PCT Filed: Nov. 30, 2022
(86) PCT No.: PCT/EP2022/083835

§ 371 Date: May 13, 2024

(30) Foreign Application Priority Data
Nov. 30, 2021 (EP) 21211223.9

(51) Int. Cl. A61M 29/00; A61B 17/22; A61M 25/00

(52) U.S. Cl. CPC A61M 29/00; A61M 25/005; A61B 17/22; A61M 25/0097; A61M 2205/0266

(57) ABSTRACT

A dilator for an endovascular treatment of a lesion within a patient includes a dilator shaft extending along a longitudinal axis (L), the dilator shaft having a braid including an arrangement of braid threads woven to form the braid. A first group of the braid threads and a second group of the braid threads are woven with one another and are arranged to cross one another at an angle (α, β) therebetween. At a first axial location of the dilator shaft the first group of the braid threads and the second group of the braid threads are arranged to cross one another at a first angle therebetween, and at a second axial location of the dilator shaft the first group of the braid threads and the second group of the braid threads are arranged to cross one another at a second angle therebetween different than the first angle.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2022/083835, which was filed Nov. 30, 2022, which application claimed priority from EP application Ser. No. 21/211,223.9, which was filed Nov. 30, 2021.

### FIELD OF THE INVENTION

The invention concerns a dilator for an endovascular treatment of a lesion within a patient, a catheter system and a method for an endovascular treatment of a lesion within a patient.

### BACKGROUND

During the past 20 years the number of endovascular devices including balloons, stents, and stent grafts, as well as adjunctive devices for debulking and true lumen reentry has exploded as industry has invested significant resources in their design and development. Additionally, when compared to earlier generations, these newer devices are made with lower profiles, different delivery shaft lengths, as well as varying guidewire platforms and delivery systems. The increase in device options has facilitated the growth of endovascular therapies. However, anatomic constraints remain the one constant in treating lesions. In other words, if one cannot reach the lesion, the lesion cannot be treated. There has been an equally phenomenal growth in guidewires, catheters, and sheaths that permit access to lesions that were previously not possible to treat.

EP 3 322 470 B1 discloses a functionally integratable catheter system CTO dilator with a reinforced shaft and a dilator.

For treating an endovascular lesion, it has been envisioned to use a catheter system including a support catheter in which a dilator is movably received such that, by advancing the dilator towards a lesion, the lesion may be treated, for example by penetrating an occlusion to free a passage through a vessel. As a fundamental principle it herein is understood that only such lesions can be treated which can be reached by the endovascular treatment device, anatomic constraints possibly hindering the advancement of a treatment device such as a dilator.

By using a dilator having a braid-reinforced dilator shaft, that is a dilator shaft having a braid including an arrangement of braid threads woven to form the braid, the dilator shaft may be designed to have a sufficient stiffness to allow for a penetration of a lesion such as a chronic total occlusion (CTO). The braid threads herein are woven and are embedded for example in a surrounding matrix material, such that a shaft is formed which may be pushed towards a lesion and may be used to penetrate the lesion by force transmission via the dilator shaft.

A chronic total occlusion (CTO) is the complete obstruction of a coronary artery. CTO having soft CTO caps in the beginning can start aging and can get hard, fibrous CTO caps with time.

### SUMMARY OF THE INVENTION

A preferred dilator according to the invention includes a dilator shaft includes a distal dilator shaft end and a proximal dilator shaft end and the dilator shaft extends along a longitudinal axis (L). The dilator shaft includes at least one braided section with an arrangement of braid threads woven to form a braid, a first group of the braid threads and a second group of the braid threads being woven with one another and being arranged to cross one another at an angle therebetween. The angle varies between the distal dilator shaft end and the proximal dilator shaft end, and the dilator shaft includes at least one longitudinal wire extending longitudinally along the dilator shaft. The at least one longitudinal wire is interlaced with the first group of the braid threads and the second group of the braid threads.

The preferred dilator, a catheter system and a method for an endovascular treatment of a lesion within a patient allow for an improved steerability and pushability of the dilator towards a lesion within a patient in order to provide for a treatment of the lesion.

### BRIEF DESCRIPTION OF THE DRAWINGS

FIG. 1 shows an embodiment of a multi-functional catheter system;

FIG. 2 shows an embodiment of a support catheter;

FIG. 3 shows an embodiment of a dilator;

FIG. 4A shows an embodiment of a dilator;

FIG. 4B shows an enlarged view of sections of the dilator;

FIG. 5A shows a view of yet another embodiment of a dilator;

FIG. 5B shows an enlarged view of sections of the dilator; and

FIG. 6 shows a schematic, cross-sectional view of the dilator of FIGS. 5A, 5B.

### DETAILED DESCRIPTION OF THE PREFERRED EMBODIMENTS

A preferred dilator shaft has at least one braided section including an arrangement of braid threads woven to form a braid, wherein a first group of the braid threads and a second group of the braid threads being woven with one another and being arranged to cross one another at an angle therebetween, wherein the angle varies between the distal dilator shaft end and the proximal dilator shaft end. Optionally the dilator shaft further has at least one non-braided section including no braid threads. A non-braided section may be arranged between two braided sections.

At a first axial location of the dilator shaft the first group of the braid threads and the second group of the braid threads are arranged to cross one another at a first angle therebetween, and at a second axial location of the dilator shaft the first group of the braid threads and the second group of the braid threads are arranged to cross one another at a second angle therebetween different than the first angle.

The dilator shaft is reinforced by a braid which is formed by an arrangement of braid threads which are woven to form the braid. To form the braid, herein, a first group of the braid threads is woven with a second group of the braid threads, such that the braid threads of the first group and the braid threads of the second group are arranged at an angle with respect to one another. Hence, the threads of the first group and the threads of the second group are interlaced to form the woven braid. In particular, the braid threads may each extend circumferentially about the longitudinal axis, wherein the braid threads of the first group and the braid threads of the second group are arranged at opposite inclination angles such that the braid threads of the first group and the braid threads of the second group cross each other and are interlaced to form a braided mesh.

The braid may in particular form a tubular structure extending longitudinally along the dilator shaft and hence may be circumferentially closed, the braid being formed by braid threads circumferentially extending about the longitudinal axis at a prescribed inclination, the braid threads of the first group and the braid threads of the second group having opposite inclinations such that the braid threads of the first group and the braid threads of the second group cross each other in an interlaced fashion to form the woven braid.

Herein, the threads of the braid do not cross at a constant angle when viewed along the longitudinal axis of the dilator shaft. Rather, at different axial locations the first group of the braid threads and the second group of the braid threads are arranged at different angles with respect to one another. In particular, at a first axial location the first group of the braid threads and the second group of the braid threads are arranged to cross one another at a first angle therebetween, and at a second axial location the first group of the braid threads and the second group of the braid threads are arranged to cross one another at a second angle therebetween different than the first angle.

Thus, one embodiment concerns a dilator for an endovascular treatment of a lesion within a patient, including: a dilator shaft having a distal dilator shaft end and a proximal dilator shaft end and the dilator shaft extending extends along a longitudinal axis, the dilator shaft having a braid including an arrangement of braid threads woven to form the braid, a first group of the braid threads and a second group of the braid threads being woven with one another and being arranged to cross one another at an angle therebetween, wherein at a first axial location of the dilator shaft the first group of the braid threads and the second group of the braid threads are arranged to cross one another at a first angle therebetween, and at a second axial location of the dilator shaft the first group of the braid threads and the second group of the braid threads are arranged to cross one another at a second angle therebetween. At the first axial location of the dilator shaft, e.g. at the distal dilator shaft end, the first angle may be larger than the second angle at the second axial location of the dilator shaft, e.g. at the proximal dilator shaft end.

As the angle in between the crossing groups of the braid threads preferably varies along the longitudinal axis of the dilator shaft, the flexibility and pushability characteristics of the dilator shaft vary along the longitudinal axis. By adjusting the angle in between the crossing braid threads, the dilator shaft may be defined such that the dilator shaft in one region includes an increased flexibility, whereas in another region the dilator shaft includes an increased axial stiffness and hence improved pushability. A larger pitch angle of the braid enables higher flexibility, whereas a smaller pitch angle enables more pushability. For example, close to a distal end the dilator may include an increased flexibility, whereas farther remote from the distal and the dilator may include an increased axial stiffness in order to facilitate a force transmission by the dilator. This a first (pitch) angle, hereinafter named α, at the distal dilator shaft end is larger than a second (pitch) angle, hereinafter named β, at the proximal dilator shaft end. For example, the first pitch angle is 100° and the second pitch angle is 90°. The pitch angle is the angle between a first braid thread and a second braid thread, crossing the first braid thread. Such a dilator enables optimal push and force transmission. Furthermore, the steerability and flexibility of the dilator shaft is enhanced which allows the physician individual support of the guidewire during access to the lesion.

In one embodiment, the angle at which the groups of the braid threads cross each other may vary between the first axial location and the second axial location. The angle may vary continuously or gradually, for example continuously increase, continuously decrease, gradually increase or gradually decrease, between the first axial location, e.g. the distal dilator shaft end, and the second axial location, e.g. the proximal dilator shaft end.

In another embodiment, the dilator shaft may include different sections, wherein within each section the braid threads of the different groups are arranged to cross each other at a particular angle.

For example, in one embodiment the dilator shaft includes a first section having a first axial length and a second section having a second axial length, wherein in the first section the first group of the braid threads and the second group of the braid threads are arranged to cross one another at the first angle therebetween, and in the second section the first group of the braid threads and the second group of the braid threads are arranged to cross one another at the second angle therebetween. Hence, in different sections the braid threads of the braid are arranged at different angles with respect to one another, such that in different sections different flexibility and stiffness (pushability) characteristics are established.

The sections have a finite length larger than 0. The sections herein beneficially join one another along the longitudinal axis, the first section for example being formed at a distal end of the dilator shaft and the second section proximally adjoining the first section.

In one embodiment, the dilator shaft includes a third section having a third axial length, wherein in the third section the first group of the braid threads and the second group of the braid threads are arranged to cross one another at a third angle therebetween different than the first angle and the second angle. Hence, in a third section the different braid threads may be arranged at another, third angle with respect to one another, such that in the third section different flexibility and pushability characteristics as compared to the first section and the second section are established.

The braid threads may for example be formed by wires, for example metal or metal alloy wires, such as stainless-steel wires or nitinol wires.

In one embodiment, the dilator shaft includes a matrix material in which the braid is embedded. The matrix material may for example be a polymer material, such as a polypropylene material, a polyethylene material, an FEP material, or an ETFE material.

In one embodiment, the dilator shaft includes an inner dilator lumen longitudinally extending along the dilator shaft. Herein, one or multiple lumens may be provided within the dilator shaft, wherein a dilator lumen may for example be used to advance a contrast agent or another fluid through the dilator towards a lesion, or to guide the dilator along a guidewire. The dilator lumen beneficially is arranged radially within the braid, such that the braid circumferentially surrounds the lumen.

In one embodiment, the dilator shaft includes one or multiple longitudinal wires extending longitudinally along the dilator shaft, preferably running from the distal dilator shaft end to the proximal dilator shaft end. For example, a multiplicity of pairs of longitudinal wires may be provided and may extend longitudinally along the dilator shaft, the longitudinal wires beneficially being interlaced with the first group of the braid threads and the second group of the braid threads such that the longitudinal wires are woven into the braid formed by the first group of the braid threads and the second group of the braid threads.

In case multiple pairs of longitudinal wires are provided, a wire of a pair of longitudinal wires may be placed at a prescribed circumferential location in immediate proximity to its paired longitudinal wire, the different pairs of longitudinal wires being displaced equidistantly with respect to one another along the circumferential direction. Within a pair of longitudinal wires the distance between two wires forming the pair of wires, is less than 50 μm, preferably less than 30 μm. Adding n-pairs of longitudinal wires, where n is 1 to 10, preferably 2 to 4, offers an improved shapability of the dilator shaft. The dilator shaft may have a diameter of less than 1 mm, preferably between 0.5 mm and 1 mm.

The longitudinal wires may for example be formed by metal or metal alloy wires, such as stainless-steel wires or nitinol wires. The longitudinal wires may alternatively be formed by polymer wires.

Thus, a dilator for an endovascular treatment of a lesion within a patient is disclosed, including a dilator shaft having a distal dilator shaft end an a proximal dilator shaft end and the dilator shaft extends along a longitudinal axis, either having a braided section or having no braided section, wherein the dilator shaft includes at least one longitudinal wire, preferably a multiplicity of pairs of longitudinal wires, extending longitudinally along the dilator shaft, and preferably running from the distal dilator shaft end to the proximal dilator shaft end.

In another aspect, a catheter system includes a support catheter forming a support catheter lumen and a dilator of the kind described above, the dilator being received in the support catheter lumen and being movable within the support catheter lumen.

For treating a lesion, the dilator may be inserted into the support catheter lumen and may be advanced towards a lesion in order to penetrate e.g. a chronic total occlusion, wherein the dilator may be moved through the support catheter lumen such that it extends and protrudes from a distal end of the support catheter to penetrate the chronic total occlusion, or may be advanced together with the support catheter such that the dilator together with the support catheter is used to penetrate the chronic total occlusion.

The dilator can be used to reach or to get access to a lesion in a human or animal body to be treated.

The dilator may be used in a catheter system. The catheter system may be a multi-functional catheter system or interventional catheter system. Multi-functional catheter system means that the support catheter can be accommodated simultaneously or consecutively with different inner members, like the dilator and a guidewire and/or balloon catheter (e.g. a percutaneous transluminal angioplasty (PTA) balloon catheter or a percutaneous transluminal coronary angioplasty (PTCA) balloon catheter).

A multi-functional catheter system includes a support catheter and at least one, preferably one, support catheter lumen.

The support catheter may include a locking handle which is configured in a restricted moving state to lock the axial movement of the dilator with respect to the support catheter and which is configured in an unrestricted moving state to unlock the axial movement of the dilator from the support catheter such that the dilator may be moved with respect to the support catheter. The locking handle may include an axial movement restriction element including an actuation mechanism and a locking mechanism, wherein the axial movement restriction element is capable of restricting an axial movement of the dilator in a restricted moving state compared to the axial movement in the unrestricted moving state. The locking handle may be arranged at a proximal support catheter end or at a support catheter shaft.

The dilator shaft extends between a distal dilator end and a proximal dilator end, and wherein the distal dilator end has a proximal segment, a distal segment, and optionally one or more intermediate segments being arranged between the proximal segment and the distal segment. The distal dilator end may be connected or connectable to the dilator shaft. The proximal segment may be connected to or connectable to the dilator shaft. The distal segment may have a uniform radial circumference and the proximal segment may have a uniform radial circumference, and the radial circumference of the distal segment may be smaller than the radial circumference of the proximal segment.

In yet another aspect, a method for an endovascular treatment of a lesion within a patient is provided, the method including: providing a support catheter of a catheter system, the support catheter forming a support catheter lumen; and inserting a dilator of the catheter system into the support catheter lumen, the dilator including a dilator shaft extending along a longitudinal axis, the dilator shaft having a braid including an arrangement of braid threads woven to form the braid, a first group of the braid threads and a second group of the braid threads being woven with one another and being arranged to cross one another at an angle therebetween, wherein at a first axial location of the dilator shaft the first group of the braid threads and the second group of the braid threads are arranged to cross one another at a first angle therebetween, and at a second axial location of the dilator shaft the first group of the braid threads and the second group of the braid threads are arranged to cross one another at a second angle therebetween different than the first angle.

The advantages and advantageous embodiments described above for the dilator and the catheter system equally apply also to the method, such that it shall be referred to the above in this respect.

A support catheter and a dilator arranged in the support catheter lumen can be used as crossing catheter system. A catheter system including a dilator in particular enables a crossing of a CTO having soft CTO caps using only the dilator for crossing, but also enables a crossing of hard, fibrous CTO caps using the support catheter together with the dilator for crossing the CTO. The support catheter works in tandem with the dilator, providing additional column strength and increasing the stiffness (pushability) of the catheter system to move through the occlusion.

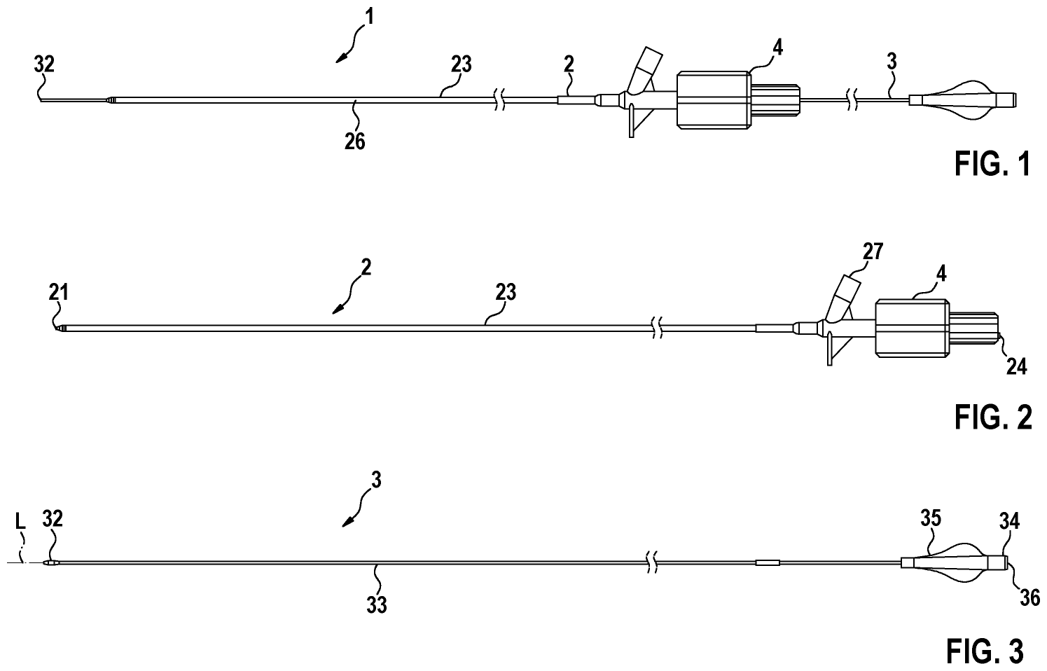

FIG. 1 shows a catheter system 1, also denoted as multi-functional catheter system, including a support catheter 2 and a dilator 3 having a distal dilator end 32. The support catheter shaft 23 defines a support catheter lumen 26 capable of receiving the dilator 3. The dilator 3 may hence be arranged within the support catheter lumen 26 of the support catheter 2. The dilator may include a locking handle 4.

Referring now to FIG. 2, the support catheter 2 includes a distal support catheter end 21, a proximal support catheter end 24 and a support catheter shaft 23 extending between the support catheter distal end 21 and the support catheter proximal end 24. At the proximal support catheter end 24, a locking handle 4 is arranged. The locking handle 4 may be user actuatable and may serve, in a locked position, to lock the dilator 3 with respect to the support catheter 2 and, in an unlocked position, to unlock the dilator 3 from the support catheter 2 such that the dilator 3 may be moved with respect to the support catheter 2.

The support catheter 2 may include one or more support catheter ports 27, preferably one or more ports for injecting or withdrawing a fluid, e.g. a flushing port, an inflation port and/or a deflation port.

The distal support catheter end 21 is designed to be inserted into a human or animal body for conducting an endovascular treatment. The proximal outer catheter end 24 is designed to remain outside of the patient during treatment and allows the handling of the catheter system 1 from outside of the patient.

Referring now to FIG. 3, the dilator 3 includes a dilator shaft 33 here forming the distal dilator end 32, which for example may have a tapered shape to allow for a penetration of a CTO at the site of a lesion. At a proximal dilator end 34 a dilator manifold 35 may be arranged, the dilator manifold 35 providing access to one or multiple dilator lumens.

For example, in one embodiment the dilator 3 includes a first lumen for receiving a guidewire and a second lumen for injecting a fluid medium, e.g. a contrast agent. In another embodiment, the dilator 3 may have only one lumen enabling the guiding of a guidewire as well as the injection of a contrast agent. The dilator manifold 35 may include one or multiple dilator ports 36 e.g. for injecting a fluid medium (e.g. a contrast agent) into one or multiple of the dilator lumens.

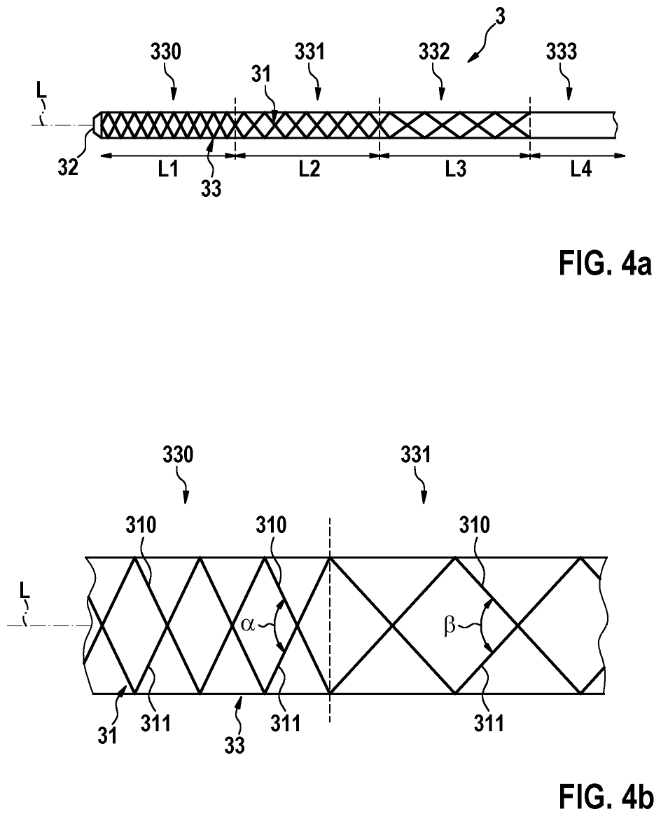

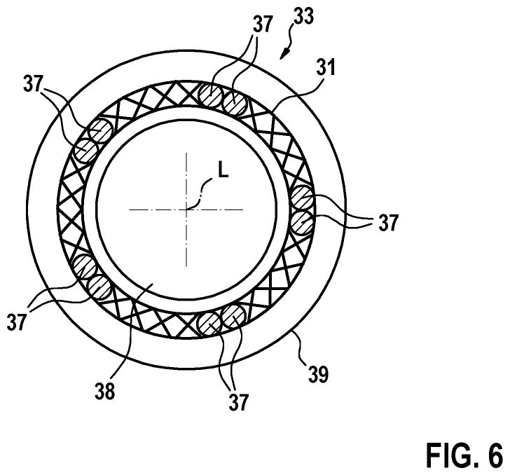

Referring now to FIGS. 4A and 4B, the dilator shaft 33 may be reinforced by a braid 31, the braid 31 being formed by woven braid threads 310, 311 which circumferentially extend about a longitudinal axis L along which the dilator shaft 33 extends. The braid 31 is embedded within a matrix material 39 (see the schematic drawing of FIG. 6), the matrix material 39 for example being formed by a polymer material, such as polypropylene, polyethylene, FEP, or ETFE.

The braid 31 is formed by woven braid threads 310, 311. Herein, two groups of braid threads are provided, the braid threads 310 of the first group extending circumferentially about the longitudinal axis at a first inclination angle, and the braid threads 311 of the second group extending circumferentially about the longitudinal axis L at a second inclination angle opposite to the first inclination angle of the braid threads 310 of the first group. The braid threads 310, 311 of the different groups are interlaced such that a woven braid 31 is formed, the braid 31 having a tubular, circumferentially closed structure and extending longitudinally along the longitudinal axis L of the dilator shaft 33.

The first group of braid threads 310 may be formed by a single wire or by multiple wires wound about the longitudinal axis L. Likewise, the second group of braid threads 311 may be formed by a single wire or by multiple wires wound about the longitudinal axis L. The braid threads 310, 311 herein are interlaced to form the woven braid 31.

As visible from FIG. 4A in view of FIG. 4B, the dilator shaft 33 includes different sections 330, 331, 332, 333, the sections 330, 331, 332, 333 adjoining each other along the longitudinal axis L. A first section 330 herein may be arranged at or close to the distal end 32 of the dilator shaft 33 and may have an axial length L1. A second section 331 may adjoin the first section 330 and may have an axial length L2. A third section 332 may adjoin the second section 331 and may have an axial length L3. A fourth section 333 may adjoin the third section 332 and may have an axial length L4.

Herein, the different sections 332, 333 differ in the structure of the braid 31.

Namely, as visible from FIG. 4B, in the first section 330 the braid threads 310, 311 may be arranged to cross one another at an angle α. The braid threads 310, 311 hence include a pitch angle±α/2 with respect to the longitudinal axis L, the pitch angle indicating the inclination of the respective braid threads 310, 311 with respect to the longitudinal axis L.

As further visible from FIG. 4B, in the second section 331 the braid threads 310, 311 cross each other at a different angle β, the angle β in the shown example being smaller than the angle α in the first section 330. The braid threads 310, 311 hence include a pitch angle±β/2 with respect to the longitudinal axis L, the pitch angle indicating the inclination of the respective braid threads 310, 311 with respect to the longitudinal axis L in the second section 331.

Generally, a large angle in between the crossing braid threads 310, 311, as in section 330, may provide for an increased flexibility in the particular section 330. The increased flexibility may enable an improved steerability in that the dilator 3 may flexibly adjust to a path to be transitioned and may be shaped by an operator for example by bending the section 330 in a desired fashion prior to inserting the dilator 3 into the support catheter 2.

A smaller angle in turn, as in the section 331, may provide for an increased axial stiffness in the particular section 331 and hence an improved pushability of the dilator 3 in that section 331.

In the adjoining, third section 332 the angle may again be different, for example smaller than the angle β in the second section 331.

In the fourth section 333 adjoining the third section 332 for example no braid 31 is provided, such that the dilator shaft 33 in the fourth section 333 is not braid-reinforced.

The braid 31 in the shown example is formed by wires, for example metal or metal alloy wires, such as stainless-steel wires or nitinol wires, which are woven with one another in order to form a braided mesh. The first group of braid threads 310 and the second group of braid threads 311 herein are arranged at different inclination angles and cross each other to form the interlaced mesh, such that the dilator shaft 33 is reinforced by a tubular, circumferentially closed wire mesh.

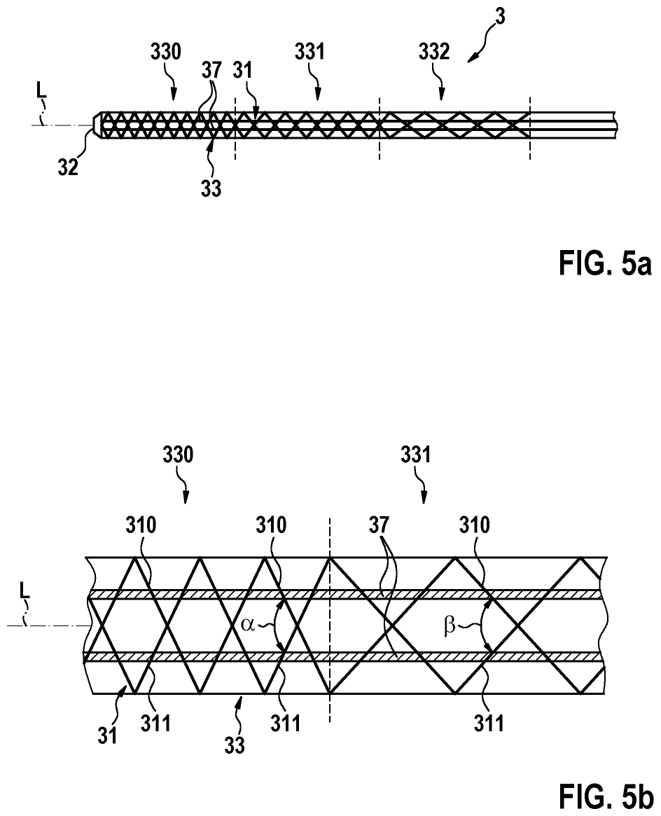

Referring now to FIGS. 5A and 5B, in another embodiment an arrangement of (pairs of) longitudinal wires 37 may be provided in addition to the reinforcement by the braid 31. The longitudinal wires 37 extend longitudinally along the longitudinal axis L (but eccentrically to the longitudinal axis L) and are interlaced with the braid threads 310, 311 of the braid 31.

In particular, as visible from FIG. 5B, the longitudinal wires 37 may cross the braid threads 310, 311 such that e.g. a particular longitudinal wire 37 is placed radially outside the braid threads 310 and radially inside the braid threads 311, as for the longitudinal wire 37 shown in FIG. 5B at the top, or vice versa, as for the longitudinal wire 37 shown in FIG. 5B at the bottom.

Referring now to FIG. 6, the longitudinal wires 37 may for example be arranged in pairs, such that a particular pair of wires 37 is arranged at an associated circumferential location and is circumferentially displaced, beneficially equidistantly displaced, with respect to other pairs of longitudinal wires 37. The longitudinal wires 37 herein are interlaced with the braid threads 310, 311 forming the braid 31, such that the longitudinal wires 37 are arranged in an interlaced fashion within the layer of the braid 31.

By providing one or multiple longitudinal (pairs of) wires 37, the axial stiffness of the dilator shaft 33 may be increased. In addition, the shapability may further be improved in that the dilator shaft 33, for example at or close to its distal end 32, may be shaped to assume a curved form.

As also schematically shown in FIG. 6, a dilator lumen 38 may longitudinally extend within the dilator shaft 33, the dilator lumen 38 beneficially being formed radially within the braid 31. One or multiple lumen 38 e.g. for receiving a guidewire or for injecting a fluid such as a contrast agent may be formed within the dilator shaft 33 and may extend longitudinally along the dilator shaft 33.

By providing a dilator shaft having a braid reinforcement exhibiting a varying angle between woven braid threads, the flexibility and stiffness may be varied along the dilator shaft. Herein, the angle in between the braid threads may be discreetly different in different sections of the dilator shaft. Alternatively, in another embodiment the angle may continuously vary between different axial locations of the dilator shaft. A continuous variation and a discrete, step-wise variation may be combined, such that in certain sections the angle in between the crossing braid threads is constant and in other sections the angle continuously varies. By adapting and varying the angle between braid threads of the braid, a flexibility versus stiffness of the dilator shaft may be adapted, such that in certain regions an increased flexibility of the dilator shaft may be established, whereas in other regions an increased stiffness for an improved force transmission may be established.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

### LIST OF REFERENCE NUMERALS

1 Catheter system
2 Support catheter
21 Support catheter distal end
23 Support catheter shaft
24 Support catheter proximal end
26 Support catheter lumen
27 Support catheter port
3 Dilator
31 Braid
310 first group of the braid threads
311 second group of the braid threads
32 Distal dilator end
33 Dilator shaft
330-333 Shaft section
34 Proximal dilator end
35 Dilator manifold
36 Dilator port
37 Longitudinal wire
38 Dilator lumen
39 Matrix material
4 Handle
α, β Angle
L Longitudinal axis
L1-L4 Length

1. A dilator for an endovascular treatment of a lesion within a patient, comprising:
a dilator shaft comprising a distal dilator shaft end and a proximal dilator shaft end and the dilator shaft extends along a longitudinal axis (L), the dilator shaft comprising at least one braided section comprising an arrangement of braid threads woven to form a braid, a first group of the braid threads and a second group of the braid threads being woven with one another and being arranged to cross one another at an angle (α, β, γ) therebetween, wherein the angle varies between the distal dilator shaft end and the proximal dilator shaft end, and wherein the dilator shaft further comprises at least one longitudinal wire extending longitudinally along the dilator shaft, wherein the at least one longitudinal wire is interlaced with said first group of the braid threads and said second group of the braid threads.

2. The dilator according to claim 1, wherein at the distal dilator shaft end the first group of the braid threads and the second group of the braid threads are arranged to cross one another at a first angle (α) therebetween, and at the proximal dilator shaft end the first group of the braid threads and the second group of the braid threads are arranged to cross one another at a second angle (β) therebetween and wherein the first angle (α) at the distal dilator shaft end is larger than the second angle (β) at the proximal dilator shaft end.

3. The dilator according to claim 1, wherein the dilator shaft comprises at least a first section comprising a first axial length (L1) and a second section comprising a second axial length (L2), wherein in said first section said first group of the braid threads and said second group of the braid threads are arranged to cross one another at said first angle (α) therebetween, and in said second section said first group of the braid threads and said second group of the braid threads are arranged to cross one another at said second angle (β) therebetween.

4. The dilator according to claim 3, wherein the second section adjoins the first section when viewed along the longitudinal axis (L).

5. The dilator (3) according to claim 3, wherein the dilator shaft comprises a third section comprising a third axial length (L3), wherein in said third section said first group of the braid threads and said second group of the braid threads are arranged to cross one another at a third angle (γ) therebetween, the third angle (γ) being different than said first angle (α) and said second angle (β).

6. The dilator according to claim 1, wherein said braid threads are formed by wires or stripes and/or, wherein said braid threads and/or the at least one longitudinal wire are made of a metal, a metal alloy or a polymer.

7. (canceled)

8. The dilator according to claim 1, wherein the dilator shaft comprises a polymer matrix material embedding said braid.

9. (canceled)

10. The dilator according to claim 1, wherein the dilator shaft comprises an inner dilator lumen longitudinally extending along the dilator shaft.

11. The dilator according to claim 10, wherein said inner dilator lumen is arranged radially within said braid.

12. (canceled)

13. The dilator according to claim 9, wherein the dilator shaft has at least one non-braided section comprising no braid threads.

14. (canceled)

15. A dilator for an endovascular treatment of a lesion within a patient, comprising:
a dilator shaft having a distal dilator shaft end and a proximal dilator shaft end and the dilator shaft extends along a longitudinal axis (L), the dilator shaft comprising at least one braided section comprising an arrangement of braid threads woven to form a braid, a first group of the braid threads and a second group of the braid threads being woven with one another and being arranged to cross one another at an angle (α, β, γ) therebetween, and at least one non-braided section comprising no braid threads, wherein the angle varies between the distal dilator shaft end and the proximal dilator shaft end.

16. The dilator according to claim 15, wherein at the distal dilator shaft end the first group of the braid threads and the second group of the braid threads are arranged to cross one another at a first angle (α) therebetween, and at the proximal dilator shaft end the first group of the braid threads and the second group of the braid threads are arranged to cross one another at a second angle (β) therebetween and wherein the first angle (α) at the distal dilator shaft end is larger than the second angle (β) at the proximal dilator shaft end.

17. The dilator according to claim 1, wherein the dilator shaft comprises at least a first section having a first axial length (L1) and a second section comprising a second axial length (L2), wherein in said first section said first group of the braid threads and said second group of the braid threads are arranged to cross one another at said first angle (α) therebetween, and in said second section said first group of the braid threads and said second group of the braid threads are arranged to cross one another at said second angle (β) therebetween.

18. (canceled)

19. The dilator according to claim 16, wherein the dilator shaft comprises a third section comprising a third axial length (L3), wherein in said third section said first group of the braid threads and said second group of the braid threads are arranged to cross one another at a third angle (γ) therebetween, the third angle (γ) being different than said first angle (α) and said second angle (β).

20-24. (canceled)

25. The dilator according to claim 16, wherein the dilator shaft comprises a polymer matrix material embedding said braid.

26. (canceled)

27. The dilator according to claim 16, wherein the dilator shaft comprises an inner dilator lumen longitudinally extending along the dilator shaft.

28-42. (canceled)

43. A multi-functional catheter system comprising a support catheter and at least one support catheter lumen, wherein the support catheter is configured to accommodate simultaneously or consecutively at least two inner members in the at least one support catheter lumen and wherein one of the at least two inner members is a dilator according to claim 1.

44. The multi-functional catheter system according to claim 43, wherein another of the at least two inner members is a guidewire or a balloon catheter.

45-47. (canceled)

48. The dilator of claim 1, wherein the at least one longitudinal wire runs from the distal dilator shaft end to the proximal dilator shaft end.

49. The dilator according to claim 15, wherein the dilator shaft comprises at least one longitudinal wire extending longitudinally along the dilator shaft.
